(19) United States
(12) Patent Application Publication
(10) Pub. No.: US 2023/0049600 A1
(43) Pub. Date: Feb. 16, 2023

(54) Housing Arrangement of a Flue Gas Filtration System Having an Integrated Option for Liquid Separation

(71) Applicant: W.O.M. World of Medicine GmbH, Berlin (DE)

(72) Inventors: Fabian Langen, Berlin (DE); Tobias Kluge, Berlin (DE); Jan Bischof, Berlin (DE); Esther Fels, Berlin (DE); Carl Chambers, Berlin (DE)

(73) Assignee: W.O.M. World of Medicine GmbH, Berlin (DE)

(21) Appl. No.: 17/785,706

(22) PCT Filed: Dec. 17, 2020
(86) PCT No.: PCT/DE2020/000338

§ 371 Date: Jun. 15, 2022

(30) Foreign Application Priority Data
Dec. 18, 2019 (DE) 102019008798.4
May 7, 2020 (DE) 102020002738.5

(51) Int. Cl. A61M 13/00

(52) U.S. Cl. CPC A61M 13/003; A61B 2018/00172

(57) ABSTRACT

Subject matter of the invention is a hose set intended for surgical interventions and having a device coupling feature, which includes an arrangement of a water trap and a filter in a single-piece housing.

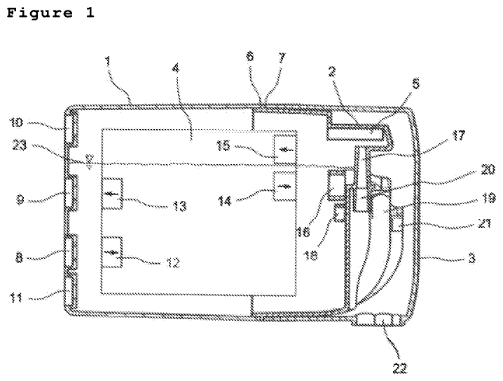

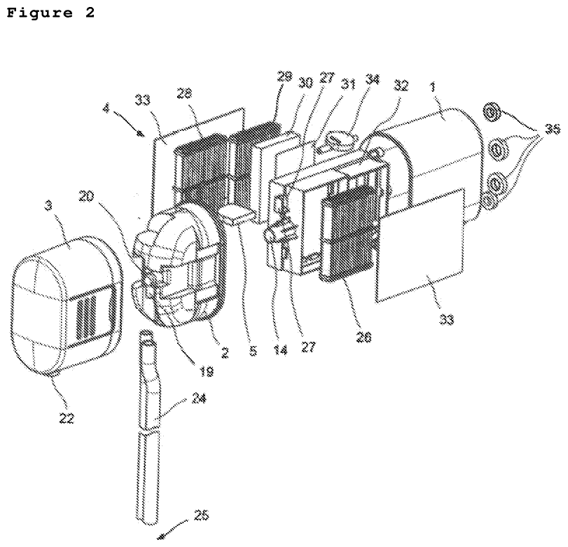

### SUBJECT MATTER OF THE INVENTION

Subject matter of the invention is a hose set intended for surgical interventions and having a device-connection feature, which hose set includes an arrangement of a water trap and a filter in a single-piece housing.

### PRIOR ART

Typically, the expansion of body cavities for minimally invasive surgical interventions occurs by means of carbon dioxide gas (insufflation). This invention is also useful for any other gaseous expansion medium.

In addition to purely mechanical instruments such as pliers or scissors, there exist further groups of surgical tools that can manipulate the tissues by means of, e.g., thermal effects. The conventional techniques include electrical surgery (also high-frequency/h.f. surgery), as well as the applications of laser beams or ultrasound (u.s.). The mentioned techniques allow cutting of tissues and a simultaneous arrest of bleeding (coagulation). By the thermal modification of the tissue, surgical flue gases are generated that, on the one hand, affect the vision of the surgeon and, on the other hand, contain a multitude of toxic constituents. The exhaust plume is an aerosol and is composed of separated cell fragments and condensation particles that should be discharged from the situs, together with the generated noxious gases. Therefore, there is a demand for a device, which sucks away the generated smoke and simultaneously filters out health-damaging substances, before the exhaust air is returned to the operating room.

It turned out that a device based on fiber or membrane filter media is not sufficient to filter the flue gases occurring during surgery. It turned out that an additional liquid separation (in the following, called water trap) is required, in particular in minimally invasive surgical interventions. Only by the separation of condensed and sucked-in liquids, the service life of fiber or membrane filter media can sufficiently be assured. The use of a water trap separate from the device-connection feature or arranged externally is material-intensive. A particular disadvantage is, however, the required positioning of the water trap along the hose and in particular in a defined orientation relative to gravity.

Heretofore, separate housings were included as external water traps in the gas path of the suction channel. For the technical solution, water traps in various designs are known, based on the principle of separation by gravity (water drops, gas raises). Further, solutions are known, in which absorbing media are employed, as well as housing arrangements that delimit, for instance, liquid from the filter media by a separation wall.

The free position of such water traps has a particularly negative effect, since raising the hose set (e.g., by the surgery nurse) may lead to that the liquid will arrive at the particle filter.

### SOLUTION ACCORDING TO THE INVENTION

For solving the above problems, the present invention teaches a device for smoke and water separation of a medical insufflator having a suction pump for desufflation, comprising:

at least one liquid-tight housing having a first connection for mechanically coupling the housing to the insufflator housing,

at least one second connection for connecting at least one desufflation hose,

at least one volatile particle filter, at least one cavity,

the gas stream leading from the patient through the first hose into the housing through the cavity and then through the volatile particle filter to the suction pump, characterized by that at least one cavity of the housing serves as a water trap.

The device according to the invention consists of a housing, to which the suction pump of an insufflator is mechanically coupled. Coupling occurs such that the orientation of the housing is fixed so that a rotation about the connecting axis is excluded. Nevertheless, the coupling allows for a fluid connection between the suction pump and the interior of the housing. For this, numerous solution options are known that need not further be discussed here.

The housing further comprises at least one second connection for connecting at least one desufflation hose, which sucks gas from the interior of the body of a patient. On the patient's side, the desufflation hose preferably includes a trocar.

The housing includes at least one conventional volatile particle filter for removing particles from the gas stream. Such filters are known to the person skilled in the art and are commercially available so here no further explanations are required. Upstream of the main volatile particle filter (flat or folded, e.g., ULPA class), a pre-filter (flat or folded, e.g., EPA class) may be arranged.

At least one cavity of the housing serves as a water trap for the separation and collection of water droplets from the gas stream. The separation occurs purely mechanically, e.g., by that the gas stream is conducted against a wall, where the drops will then move downward.

If applicable, further separation elements for water droplets or water vapor may be connected, which will be described below in more detail.

Since the housing of the device according to the invention is intended to collect the separated water, the housing will have to be made liquid-tight.

The arrangement of the components in the housing occurs such that the gas stream passes from the patient through the first hose into the housing. In the housing, the gas stream is conducted first through the cavity for the purpose of water separation. Then, the gas is conducted through the volatile particle filter to the suction pump.

The housing solution according to the invention has several advantages:

By an integration in the housing of the filter system, handling (positioning of the housing/hose unit (hose set) and correct connection to the device) is simplified, and the water trap itself is fixed in its position. By the fixed orientation of the housing, the latter is connected to the device non-rotatably in all directions, tilting etc. is not possible.

In contrast to a water trap, which delimits, for instance, by means of a separation wall the collected liquid in a cavity provided exclusively therefor, the solution according to the invention enables that the liquid surrounds the included assembly with the filter media (three-dimensionally). The volume being free in the interior of the housing is thus utilized in an optimum manner for liquid collection.

The maximization of the liquid capacity occurs by that the transition to the particle filter with fixed orientation takes place as high as possible at the top and utilizing all resulting cavities. The assembly for receiving the filter media is configured such that the fluid filling level cannot reach the media before the inner free volume is nearly completely occupied. The use of the complete available cavity requires that the housing is completely liquid-tight.

The integration of the water trap in the housing allows for the positioning of the hose connections in a horizontal as well as vertical orientation. Heretofore, the hoses exit vertically to the front of the housing, which will frequently lead to kinking and thus to at least partial occlusion. Thus, in turn, results a reduced suction and insufflation power.

The water trap may include, in addition to the utilization of gravity, further principles of action, which favor the liquid separation or condensation of the humidity contained in the sucked-off gas. Examples are an acceleration or circulation of the flow by axial/tangential cyclones, utilization of further mass inertia effects for deflecting or enforcing a defined flow (e.g., deflection by 180°, baffle wall, nozzle flows), the introduction of adsorbing or absorbing media (e.g., activated carbon, super-absorbing polymers, oil separation fleeces), utilization of turbulence washer principles, utilization of coalescence effects by wire meshes, synthetic tissues or foams. Combinations of several of the mentioned principles are also possible.

Possible fluidic interfaces/connections of the housing include, while maintaining the implementation according to the invention:

gas supply to the patient (inflow);
gas discharge from the patient (outflow, also for targeted ventilation (desufflation)/flue gas suction), wherein this lumen may also be used for increased gas supply to the patient (double insufflation), or as a negative pressure for a suction container that sucks liquids and tissue remainders from the body cavity (surgical sucker);
pressure measurement channel for real-time monitoring of the cavity pressure in the body cavity in the patient.

The housing is at the proximal end (away from the patient) of the hose set and serves for forwarding all device connections of fluidic (inflow, outflow/suction, pressure measurement channels) and electrical kinds (gas heating, temperature sensors, etc.). The hose lumens may be coupled thereto in a fixed (for instance, by adhesives) or releasable manner (for instance, by pushfit couplings or similar).

Preferably, the hose set is provided as a disposable for single use. This means, normally, there is no depletion, processing, or re-use.

Variant 1: The housing includes a filling level display through a housing being transparent at least at one location with or without a scale. Detection of filling levels by other methods is also possible, according to the invention, e.g., by a capacitive filling level detection or a filling level sensor at the device that detects the filling level, e.g., by ultrasound.

Variant 2: Depletion option of the (potentially contaminated) water trap content for extending the time of use by, for instance, a septum in the housing wall or a connector (e.g., a connection for a syringe with Luer connection, or a led-out hose with a hose clamp) with a closure option, for instance, by a check valve.

Variant 3: Construction of a housing without an insufflation channel for use as a multiply usable hose set for an “out-patient”. By means of an additional hose renewable for every patient and a separate insufflation line, the cassette for flue gas suction may remain in the device and be used without processing for multiple patients.

Further optional features:

Change option in the device for utilization of another lumen, e.g., the suction line, as an additional insufflation/supply line (double insufflation). Thus, the doubled amount of gas can be introduced without an additional lumen.

During the discontinuous insufflation, so-called measurement pauses are provided, that is, a stop of the active pressure control. In these measurement pauses, the gas supply is interrupted, and the generated equilibrium pressure in the body cavity is determined by sensors in the device. In these pauses, the abdominal pressure slightly breaks down due to corresponding leakages. Via the suction line, small gas volumes of the expansion medium can be issued as stabilization pulses for compensating the leakage. For avoiding the return of the water trap content, the water trap can be circumvented in this mode of use, e.g., by means of a hose connection in the housing. A matching position of the connection that the water trap content cannot reach is also provided according to the invention. Alternatively, a third lumen can be used that conducts, via an additional connection in the housing, the stabilization pulses into the body cavity.

When the utilization of a separate pressure measurement channel (sense line) in the form of a third or fourth lumen of the hose connection for realtime/permanent measurement of the pressure existing in the body cavity by a sensor in the device is provided, this occurs without impairment of the functions according to the invention of the housing arrangement. In a similar way acts an electrical connection to a sensor positioned in the hose set or at the end close to the patient, which sensor can be guided through the housing arrangement without the arrangement according to the invention being modified. The positioning of the interfaces occurs in regions of the housing, which enable a direct passage of the measurement channel/electrical connection from the device to the hose lumen, without penetration of the filter. The pressure measurement channel can be routed through the volume of the water trap or along the outside of the housing, needs, however, to be sealed against the environment.

The solution according to the invention of the housing arrangement enables the determination of the filling level of the water trap by a sensor system in the device (for instance, by means of capacitive sensors). This detection, in turn, enables the device to issue in time an alarm, e.g., by an alert or optical signalization, or to show the actual filling level on the device display.

By means of an additional interface to the device, the static pressure in the water trap can be determined via a sensor system. In a preferred solution, this sensor system is connected via measurement channels to sensors in the device. This measurement allows for monitoring of the differential pressure increasing during utilization via the filter media and to issue in time alerts before the suction power declines or the filter media are completely clogged.

In an optional embodiment of the invention, the water separated in the water trap may serve for humidification of the gas stream, which is supplied to the body. For this purpose, the water trap needs to be in a fluid connection to the gas supply hose. In order that no pressure equalization between the gas supply hose and the hose to the suction pump occurs, it is recommended to implement the liquid transport required in this embodiment via a wick. The wick may be made, for instance, of cotton fabric. By capillary forces, liquid can be transported via the wick. In this embodiment, for instance, a gas supply hose with humidifying device (such as described, for instance, in WO 2020074027 A1, EP 2806927 A1, EP 2804649 A1, and EP 3237046 A1) could be moved on the outside past the device according to the invention. Via gas-tight openings of the water trap, on the one hand, and of the hose, on the other hand, a wick between water trap and humidifying material of the hose can permit a water transport from the water trap to the gas supply hose. In a preferred embodiment, gas supply hose with wetting agent and water trap are accommodated in a single housing. Such an embodiment and the thus effected recycling of the used water mainly has the advantage that with longer treatment time, no water refilling of the humidification hose or the humidifying device and thus no depletion of the water trap is required. For avoiding the transfer of germs, a filter may be incorporated.

### LIST OF REFERENCES

(1) Housing half with device interfaces
(2) Housing half with hose interfaces
(3) Cosmetic housing cap with hose passage
(4) Assembly with filtration media
(5) Functional element for supporting the liquid separation
(6) Gas- and liquid-tight connection
(7) Not necessarily, gas- and liquid-tight connection
(8) Housing interface for insufflation channel
(9) Housing interface for desufflation/suction channel
(10) Housing interface for, e.g., differential pressure measurement [when not required ->closed in a gas- and liquid-tight manner]
(11) Housing interface for, e.g., real-time pressure measurement channel [when not required ->closed in a gas- and liquid-tight manner, otherwise ->connected in a gas- and liquid-tight manner to (18)]
(12) Input interface of (4) for insufflation [connected in a gas- and liquid-tight manner to (8)]
(13) Output interface of (4) for desufflation/suction [connected in a gas- and liquid-tight manner to (9)]
(14) Output interface of (4) for insufflation
(15) Input interface of (4) for desufflation/suction
(16) Input interface of (2) for insufflation [connected in a gas- and liquid-tight manner to (14)]
(17) Output interface of (2) for desufflation/suction
(18) Input interface of (2) for, e.g., real-time pressure measurement channel
(19) Output interface of (2) for insufflation [connection for (24)]
(20) Input interface of (2) for desufflation/suction [connection for (24)]
(21) Output interface of (2) for, e.g., real-time pressure measurement channel [connection for (24)]
(22) Opening in (3) for one- or multi-lumen hose (24)
(23) Maximally achievable filling level in the water trap
(24) One- or multi-lumen hose
(25) Patient interface (not shown), e.g., Luer connector(s)
(26) Insufflation filter (flat or folded)
(27) Additional flow obstacle at inlet (15)
(28) Pre-filter (flat or folded), e.g., EPA class
(29) Main volatile particle filter (flat or folded), e.g., ULPA class
(30) Adsorbing medium, e.g., activated carbon
(31) Fluid lock, e.g., PVDF, PTFE
(32) Main body of (4) for receiving the filtration media (26) to (31)
(33) Lid for gas- and liquid-tight closure of (32)
(34) Filter, e.g., for differential pressure measurement via (4)
(35) Seals for device-hose set interfaces

1. A device for smoke and water separation for use with a medical insufflator having a housing and a suction pump for patient cavity desufflation, comprising;
at least one liquid-tight housing having a first connection for mechanically coupling the housing to the insufflator housing,
at least one second connection for connecting at least one desufflation hose,
at least one volatile particle filter,
at least one cavity,
wherein a gas channel leads from the patient cavity through the first desufflation hose into the housing through the at least one cavity and then through the volatile particle filter to the suction pump, and
wherein the at least one cavity of the housing serves as a water trap.

2. The device of claim 1, comprising at least one component for additional water separation, selected from the group consisting of:
axial or tangential cyclone,
baffle wall,
gas stream deflection device for deflecting the gas stream by 90° to 180°,
nozzles,
adsorbing or absorbing media,
turbulence washer,
wire mesh, and
synthetic tissues or foams.

3. The device of claim 1, further comprising at least one pressure measurement channel for real-time monitoring of cavity pressure in the patient cavity.

4. The device of claim 1, further comprising at least one filling level display.

5. The device of claim 1, further comprising a closable opening for removing separated water.

6. The device of claim 1, further comprising at least one sensor for monitoring a remaining capacity of the volatile particle filter.

7. The device of claim 1, further comprising at least one housing opening with a wick that allows the transport of the separated water into a gas supply hose and wetting agent thereof.
